(19) United States
(12) Patent Application Publication
(10) Pub. No.: US 2013/0172696 A1
(43) Pub. Date: Jul. 4, 2013

(54) MEDICAL HAND-HELD MEASURING DEVICE WITH EXCHANGEABLE PROBE

(71) Applicant: Thomas Riesinger, Nurnberg (DE)

(72) Inventor: Thomas Riesinger, Nurnberg (DE)

(73) Assignee: NAWA HEILMITTEL GMBH, Nürnberg (DE)

(21) Appl. No.: 13/816,750

(22) PCT Filed: Aug. 24, 2011
(86) PCT No.: PCT/DE11/01655

§ 371 Date: Mar. 18, 2013

(30) Foreign Application Priority Data
Aug. 27, 2010 (DE) 202010011934.0

(51) Int. Cl. A61B 5/01; A61B 5/00; A61B 5/145

(52) U.S. Cl. CPC A61B 5/01; A61B 5/14539; A61B 5/0008
USPC 600/309; 600/549

(57) ABSTRACT

The invention relates to a medical hand-held measuring device for measuring the pH value and/or the temperature of wounds, including a housing (2) having at least one display (6) for displaying the particular measured value, operating elements, and a probe (10) provided at an end of the housing, the probe having a probe arrangement (9) for producing electrical measurement signals that correspond to the pH value to be measured and/or the temperature to be measured . The probe (3) is electrically and mechanically connected to the housing (2) in a removable manner by means of a probe-sided coupling section (10) and a housing-sided coupling section (11).

### BACKGROUND OF THE INVENTION

The invention relates to a handheld medical measuring instrument for measuring the pH value and/or the temperature of wounds.

It is an object of the invention to present a handheld measuring instrument with which the condition of wounds can be assessed quickly and reliably and which for this purpose fulfills the necessary requirements for use in the medical sector and in particular in the clinical sector.

### SUMMARY OF THE INVENTION

The medical handheld measuring instrument, according to the invention, and/or the housing of the instrument correspond in form and/or size to an electrical or electronic temperature measuring instrument (e.g. fever thermometer). The handheld measuring instrument and/or its probe or pH measuring sensor (pH meter) and the temperature sensor provided in the probe and forming at least part of the sensor arrangement have a compact design that enables, by insertion of the probe or its probe tip into the wound, fast and reliable measurement of the wound's pH value and temperature.

The invention is based on the knowledge that acute wounds not requiring further special treatment have a pH value between approximately 2.8 and 3.3, whereas a pH value higher than 4.5, especially a pH value between 5 and 8, indicates a chronic wound requiring special treatment, for example, surgical treatment for removal of infected and/or necrotic tissue.

In the case of the handheld measuring instrument, according to the invention, the respective probe is provided on the handheld measuring instrument or the housing in a mechanically and electrically removable manner, so that each probe can be sterilized after use and/or disposed of as a disposable probe. Preferably, a plurality of probes are used on the same handheld measuring instrument, which probes then are provided with a probe identification that can be read by the handheld measuring instrument or an electronic measuring and analysis circuit, so that the respective probe connected to the handheld measuring instrument is uniquely identified. This also makes it possible to calibrate the sensor arrangement and/or the corresponding sensors of the respective probe connected to the handheld measuring instrument and to store the corresponding data specific to the respective probe in a memory of the electronic measuring and analysis circuitry, i.e. together with the respective probe identification and to use this calibration data respectively for analysis of the measured values from the respective probe.

Further embodiments, advantages and possible applications of the invention are disclosed by the following description of exemplary embodiments and the drawings. All characteristics described and/or pictorially represented, alone or in any combination, are subject matter of the invention.

### BRIEF DESCRIPTION OF THE DRAWINGS

The invention is described below based on exemplary embodiments with reference to the drawings, in which:

FIG. 1 shows a schematic view of a medical handheld measuring instrument according to the invention; and

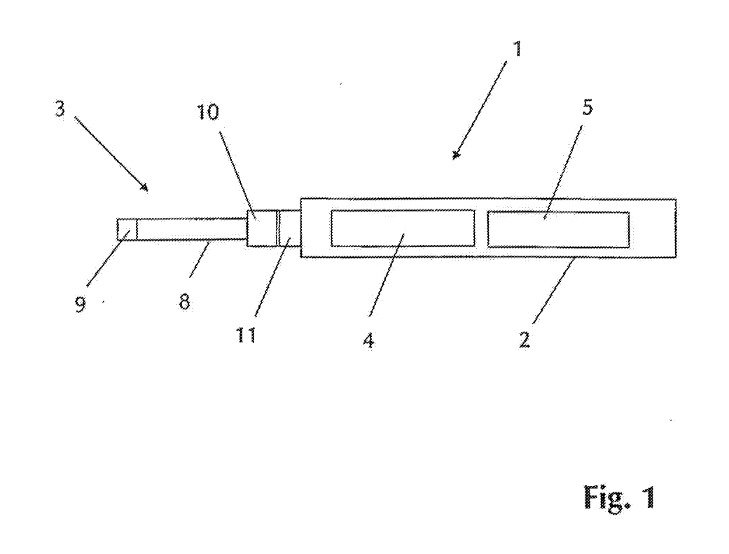

FIG. 2 shows a simplified block diagram of the medical handheld measuring instrument of FIG. 1.

### DETAILED DESCRIPTION OF THE INVENTION

The medical handheld measuring instrument comprises an elongated housing 2 and a measuring probe 3, which can be connected and disconnected mechanically and electrically on the instrument housing 2. An electronic circuit 4 and a preferably rechargeable battery 5 are accommodated inside the instrument housing 2. On the outer surface of the housing 2 is a display 6, in particular for displaying the operating state and the measurement results measured with the handheld measuring instrument 1, as well as at least one manually operated input unit, for switching the instrument on and off and for starting or calling up different functions of the handheld measuring instrument 1. In detail, the handheld measuring instrument 1 is designed for measuring the pH value and the temperature of wounds. The probe 3 comprises for this purpose of an elongated probe body 8, which is provided on one end with a sensor arrangement 9 for the measurement of the pH value and for the temperature measurement. On the other end, the probe body 9 forms the coupling section 10, with which the probe 3 can be connected mechanically and also electrically to the elongated housing 2 or to a coupling section 11 there, so that the respective measured value of the sensor arrangement 9 is transferred from the probe 3 to the electronic circuit 4 accommodated in the housing 2 and processed there in the manner described in more detail below.

The sensor arrangement 9 comprises in the depicted embodiment, of the components that enable sterilization of the probe 3, for example (c)f an electric resistor, whose electrical properties change based on the temperature and of a suitable pH sensor. The probe body 8 also includes a material that enables sterilization of the probe 3.

A plurality of probes 3 of the same type are provided for the handheld measuring instrument 1. The handheld measuring instrument 1 is further designed so that its functions are only switched on if a probe 3 or its coupling sections 10 are properly connected, for example, by means of a plug-in or bayonet connection, mechanically and electrically to the coupling section 11. Each probe 3 is provided with an electric encoding or probe identification that uniquely identifies the probe and can be detected by the electronic circuit 4. The calibration of each probe 3, or its sensor arrangement 9, takes place within the electronic circuit 4, namely in the manner that the calibration associated with the respective probe 3 is stored together with the probe identification in the electronic circuit 4.

The handheld measuring instrument further features the following parameters:

The pH measurement range extends from 4 to 10. The measurement accuracy and display of the pH value take place in increments of 0.1.
The pH measuring time, i.e. the time needed for the pH measurement, is approximately 5 seconds or less.

The temperature measurement range extends from approximately 20 to 40° C. The display takes place in increments of 0.1° C.

The calibration of the respective probe 3 and/or its sensor arrangement 9 is highly stable, so that a recalibration is necessary no more than once per day. The probe 3 and also the probe tip comprising the sensor arrangement 9 is composed of a biologically and dermatologically compatible material, which is resistant e.g. to proteins and their reaction products.

The handheld measuring instrument 1 is further designed for wireless remote data transfer, in particular, for remote data transfer of the respective measured data, together with additional data needed for security, control and/or analysis of the measured data.

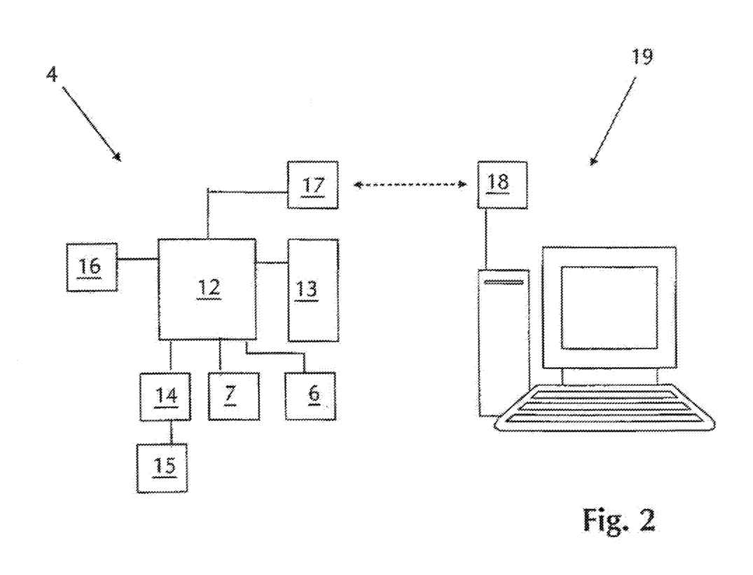

As a result of the above properties, the handheld measuring instrument 1 and/or its electronic circuit 4 are designed in the manner depicted in FIG. 2. The central unit of the electronic circuit 4 is a microprocessor 12, with which, for example, the following are associated.

A memory 13 is provided which is used for program and data storage, and in which also the measured values detected by the sensor arrangement 9 and/or processed in the processor 12 as well as the calibration of the respective probe 3 are stored together with the probe identification. The following are further associated with the processor 12: the at least one display 6, the input unit 7, a tone generator 14 with a speaker 15 for generating the above-mentioned acoustic signal, and an interface 15 for connecting the processor 12 with the respective probe 3, especially also for conversion of the electric measured signals into digital signals delivered to the processor 12 that can be processed in the latter as well as for recognition of the probe identification. The processor 12 is further associated with a transmitting and receiving device 17 for wireless communication, in particular for wireless data transfer between the handheld measuring instrument 1 and a spatially distant transmitting and receiving device 18 of a computer-supported registration and monitoring system 19.

By means of the data transfer link formed between the transmitting and receiving devices, 17 and 18, the measured value (pH value and temperature) of a monitored wound measured with the handheld measuring instrument 1 with a particular probe 3 is forwarded probe- and/or patient-specific to the registration and/or monitoring system 12 and analyzed and/or stored patient-specific in the system to determine the further therapy and/or to determine the success of the treatment.

The invention was described above based on an exemplary embodiment. It goes without saying that numerous modifications and variations are possible, without abandoning the underlying inventive idea on which the invention is based.

In addition to the optical display 6, the handheld measuring instrument preferably also features an acoustic display, which generates an acoustic signal in the event of a change in the operating state of the handheld measuring instrument and/or in case of obvious incorrect measurements.

### REFERENCE LIST

1 handheld measuring Instrument

2 housing

2.1 housing end

3 probe

4 electronic circuit

5 battery

6 display

7 input unit

8 probe body

9 sensor arrangement

10,11 coupling section

12 processor

13 memory

14 tone generator

15 speaker

16 interface

17,18 transmitting and receiving device

19 registration and/or monitoring system

What is claimed is:

1. A medical handheld measuring instrument for measuring a pH value or a temperature of wounds, comprising a housing with at least one display for displaying a respective measured value and with control elements, and the handheld measuring instrument comprising a probe provided on an end of the housing with a sensor arrangement for generating electrical measured signals corresponding to a measured pH value or a measured temperature, wherein the probe is mechanically and electrically removably connected to the housing by of a sensor-side coupling section on a housing-side coupling section.

2. The medical handheld measuring instrument according to claim 1, comprising a plurality of like probes connected mechanically and electrically by the sensor-side coupling section with the housing-side coupling section of the handheld measuring instrument.

3. The medical handheld measuring instrument according to claim 1, wherein the probe is sterilizable or usable as a disposable probe.

4. The medical handheld measuring instrument according to claim 1 wherein, the probe is provided with probe encoding or identification for identifying and differentiating the probe from other probes of the same type, and a processor is provided in the housing for detecting or reading the probe encoding.

5. The medical handheld measuring instrument according to claim 4, wherein an electronic circuit comprising the processor is provided in the housing for analysis or display of the electrical measured signals delivered by the probe.

6. The medical handheld measuring instrument according to claim 5, further comprising an electronic memory for storing a calibration of the sensor arrangement of the probe based on probe identification.

7. The medical handheld measuring instrument according to claim 1, further comprising at least one acoustic signal generator.

8. The medical handheld measuring instrument according to claim 1, further comprising; wireless data communication between the handheld measuring instrument and the display accommodated in the housing and analysis electronics and a system that is spatially remote from the handheld measuring instrument, for a registration or monitoring system or a transmitting or receiving device of the system.
